(19) United States
(12) Patent Application Publication
(10) Pub. No.: US 2011/0196416 A1
(43) Pub. Date: Aug. 11, 2011

(54) COMPRESSION CLOTHING

(76) Inventor: Bodo W. Lambertz, Pfaffikon (CH)

(21) Appl. No.: 12/998,439

(22) PCT Filed: Oct. 26, 2009
(86) PCT No.: PCT/EP2009/007633

§ 371 Date: Apr. 19, 2011

(30) Foreign Application Priority Data
Oct. 24, 2008 (DE) 20 2008 014 202.4

(51) Int. Cl. A61B 17/00

(52) U.S. Cl. 606/201

(57) ABSTRACT

The invention relates to compression clothing (1) for wearing on the skin (2), made of elastically resilient material. The clothing (1) is provided in some regions with means (3, 8) for compression.

The invention pertains to compression clothing to be worn on the skin, made of elastically resilient material.

Compression clothing generates external pressure on the body; in the case of compression stockings, for example, pressure is exerted on the tissues of the enclosed leg. Compression stockings are manufactured in such a way that the pressure which is exerted increases from top to bottom, similar to the increase in tissue pressure and thus acting in the same direction as gravity. The pressure exerted by compression clothing can be selected as a function of the type of treatment desired. The pressure exerted by the clothing is divided into various compression classes.

The use of compression clothing can be divided into medical applications and cosmetic or preventive applications. The medical applications pertain to the treatment of, for example, varicose veins, leg vein thromboses, and the like. The preventive applications, in which the compression clothing exerts only a small amount of pressure on the body, pertain to the treatment of air travel thromboses, for example, or to the provision of support for people whose occupations demand that they stand for long periods of time. The preventive application can significantly reduce the risk of a thrombosis during long-distance flights, for example. In addition, compression clothing, especially compression stockings, are used in sports activities such as Nordic walking and marathon races.

The known compression clothing exerts pressure on a continuous two-dimensional area of the body. Although it is true that the pressure is distributed uniformly, the blood supply is constricted, which can, of course, be desirable in certain medical applications. In the case of preventive applications, however, especially in the area of sports, such constriction is not desirable for the following reason:

As a result of muscular work, the temperature of the muscles increases considerably during (endurance) exercises. The blood carries the heat thus produced away from the muscle and into the organism, as a result of which the core body temperature also increases. The capillaries under the skin expand as a result of the increase in temperature. This explains why, for example, the skin of the face turns red during vigorous exertion. To reduce the temperature of the blood, the body redistributes it. Oxygen-rich blood is shifted from the center of the body to the periphery, that is, toward the skin, where a cooling effect thus takes place. The blood functions, as it were, like a coolant for the body. The continuous two-dimensional area of compression produced by the known compression clothing acts negatively in this respect, because, as a result of the compression, the walls of the capillaries are squeezed together, which has the effect of slowing down the transfer of blood. The inadequate cooling which results from uniform two-dimensional compression leads in turn to a decrease in athletic performance.

This is the point at which the invention aims to provide a remedy. The invention is based on the goal of creating compression clothing which retains the advantages of the known compression clothing but which also maintains the blood supply in such a way that adequate circulation through the affected muscles is ensured. According to the invention, this goal is achieved in that compression means are provided only in certain regions.

The invention thus creates compression clothing which, although it exerts pressure on the skin, limits the blood supply to the muscles in a way that still allows a high level of performance. The reason for this is that only certain regions are subjected to compression. In the regions adjacent to the compression means, no compression takes place, which means that the blood can circulate here without any interference. In this way, the stressed muscles can be supplied with the “cooled” blood being transported from the inner areas of the organism. In spite of the compression exerted on the skin, the cooling capacity of the body remains preserved. The ability of the athlete to perform is therefore significantly improved.

The compression means are advantageously formed by strips, which are provided on the side facing the skin. The strips offer a simple means of producing regional compression. In addition, they can be arranged anywhere on the article of clothing.

In an elaboration of the invention, the strips are produced by increasing the thickness of the material. This is a simple way of producing the strips. When a woven fabric is used as the material, the increase in the material thickness can be achieved through the use of threads of larger diameter or possibly by increasing the number of threads.

In another elaboration of the invention, the strips have an approximately triangular form [in cross section]. This ensures that only a very narrow section of the strip rests on the skin. This further improves the ability of the article of clothing to exert pressure only on certain regions.

It is advantageous for the strips to be arranged at regular intervals. This leads to a symmetric design of the article of clothing, which increases the effectiveness of the article of compression clothing and also simplifies its production.

The strips of the compression clothing can also be provided with a coating. Various materials can be used as a coating for the purpose of achieving different effects. For example, antifungicidal or antibacterial effects can be achieved through the choice of suitable coatings. It is also possible to achieve friction-reducing effects through the effective choice of a coating.

According to another embodiment of the invention, the compression means are formed by fabric bands, the elasticity of which differs from that of the basic fabric of the article of clothing. As a result, it is again possible to achieve regional compression. At the same time, the article of clothing has a flat structure, which is desirable under certain conditions of use.

Another improvement in the supply of “cooled” blood to the stressed muscles can be achieved by providing the compression means with interruptions. This makes it possible for blood to circulate without hindrance not only into the regions adjacent to the compression means, i.e., regions where no compression is occurring, but also into the regions where the interruptions are, where again there is no compression. The athlete's performance is thus improved even more.

Other elaborations and embodiments of the invention are described in the remaining subclaims. An exemplary embodiment of the invention is illustrated in the drawing and described in detail below:

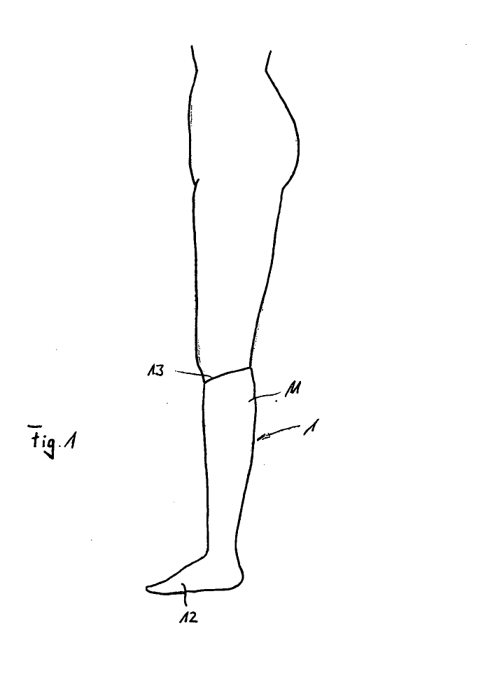

FIG. 1 shows a side view of an article of compression clothing in the form of a knee stocking, as it would appear on a human body;

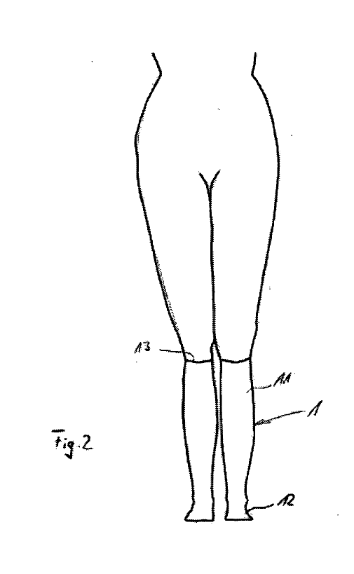

FIG. 2 shows a front view of the article of compression clothing illustrated in FIG. 1;

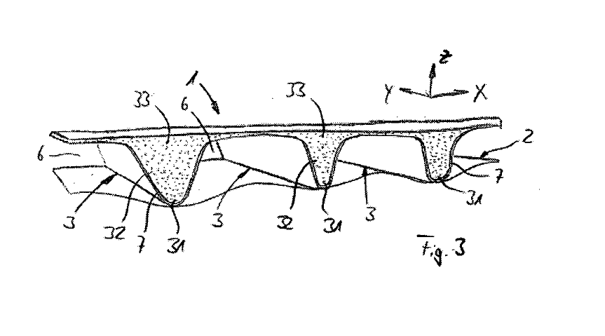

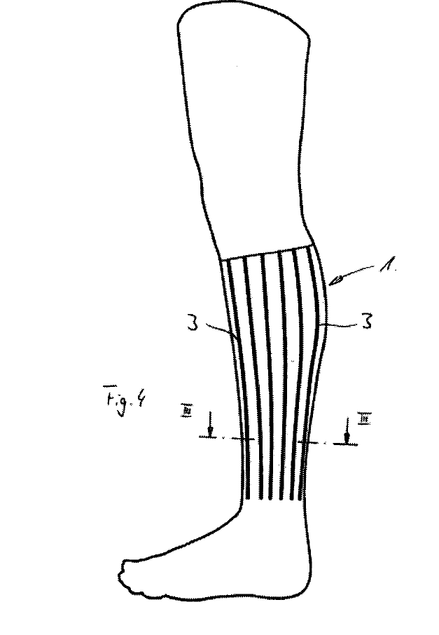

FIG. 3 shows a partial cross-sectional diagram taken along line BI-BI through the article of compression clothing shown in FIG. 4;

FIG. 4 shows a schematic diagram of an arrangement of strips with a vertical orientation;

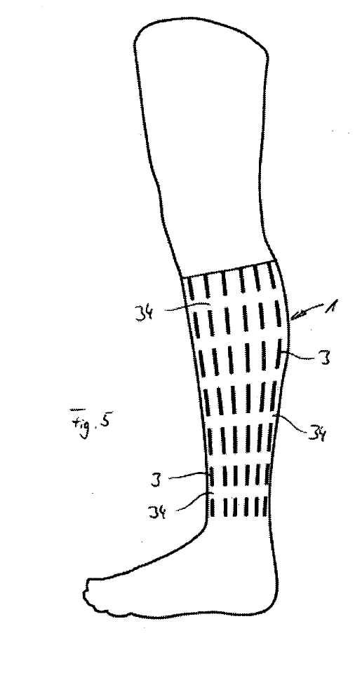

FIG. 5 shows a schematic diagram of an arrangement of strips with a vertical orientation and with interruptions;

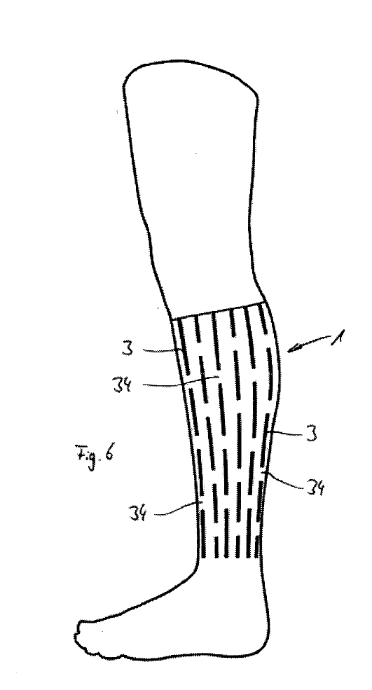

FIG. 6 shows a schematic diagram of an arrangement of strips with a vertical orientation with interruptions of a different type;

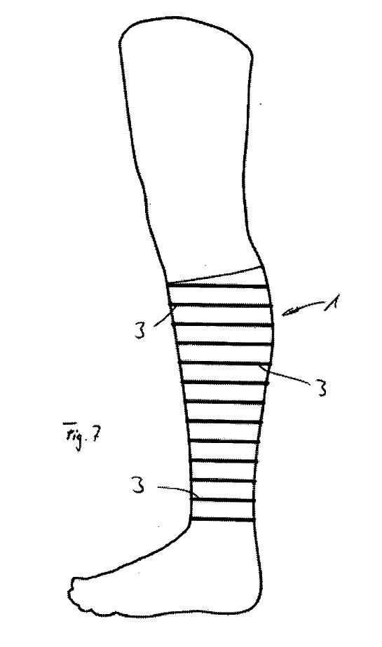

FIG. 7 shows a schematic diagram of an arrangement of strips with a horizontal orientation;

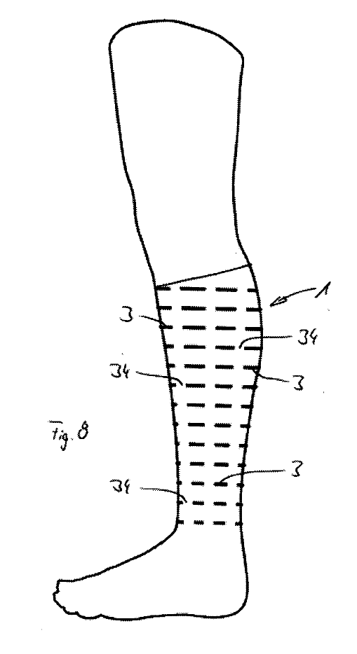

FIG. 8 shows a schematic diagram of an arrangement of strips with a horizontal orientation and with interruptions;

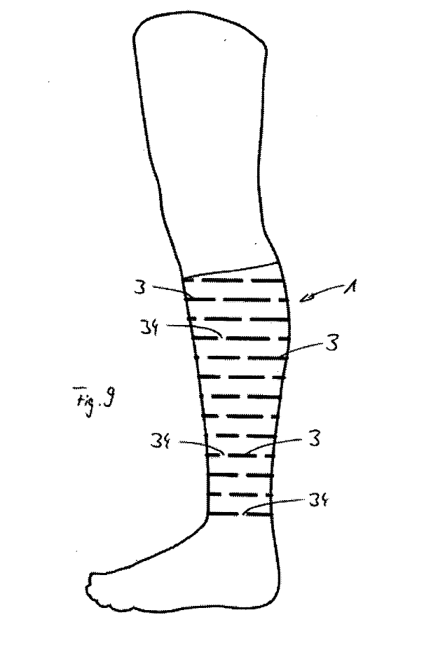

FIG. 9 shows a schematic diagram of an arrangement of strips with a horizontal orientation and with interruptions of a different type;

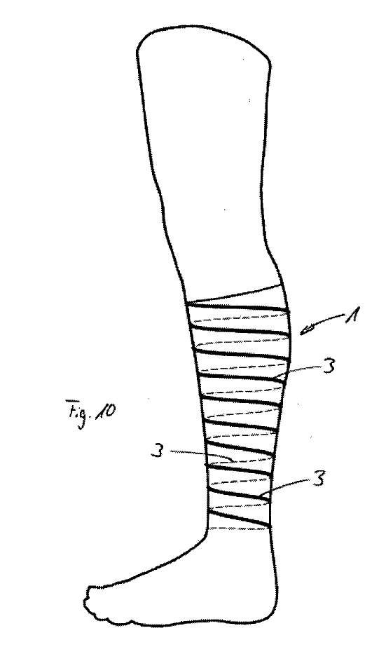

FIG. 10 shows a schematic diagram of an arrangement of strips with a spiral orientation;

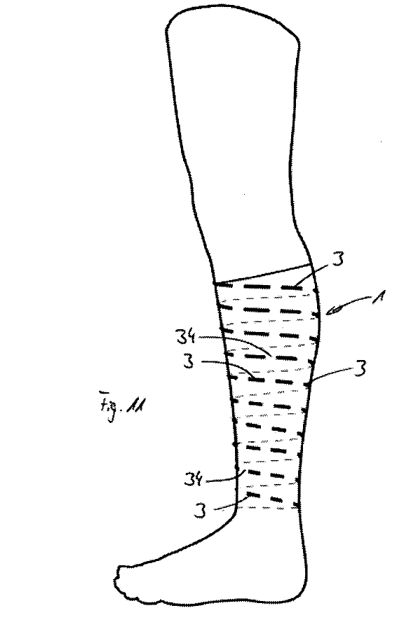

FIG. 11 shows a schematic diagram of an arrangement of strips with a spiral orientation and with interruptions;

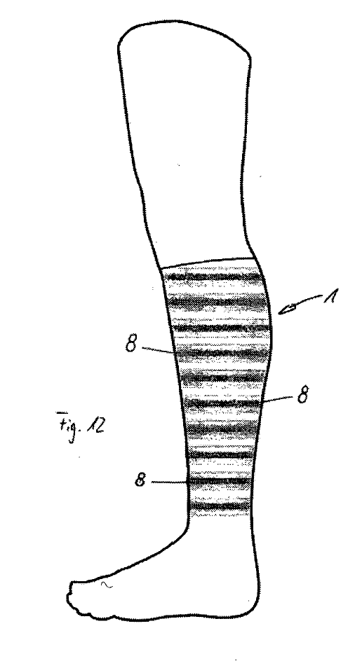

FIG. 12 shows a schematic diagram of an arrangement of fabric bands with a horizontal orientation;

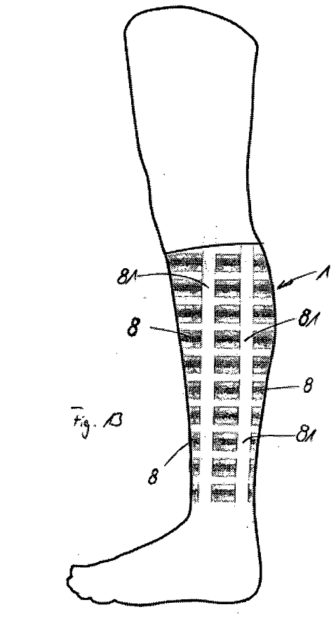

FIG. 13 shows a schematic diagram of an arrangement of fabric bands with a horizontal orientation and with interruptions;

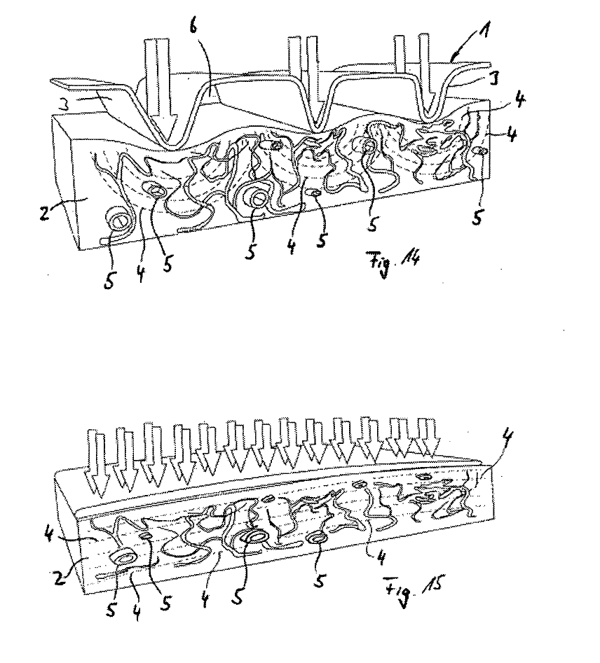

FIG. 14 shows partial schematic diagram of the regional compression effect of an article of compression clothing according to the present invention; and

FIG. 15 shows a partial schematic diagram of the uniform two-dimensional compression effect of an article of compression clothing according to the prior art.

The article of compression clothing 1 selected as the exemplary embodiment is designed in the form of a knee stocking, which is placed on the lower part of a human leg. The knee stocking comprises a shaft 11 and a foot part 12. A collar 13 is provided at the end facing away from the foot part 12.

Compression means are provided in certain regions of the article of compression clothing 1. No compression takes place in the areas adjacent to the compression means. Blood is free to circulate unhindered in these regions.

In the case of the article of compression clothing according to FIGS. 3-11, the compression means are formed by strips 3, which are provided on the side facing the skin 2. In the present exemplary embodiment, the strips 3 are arranged at regular intervals. As can be seen in FIG. 3, the strips 3 are produced by increasing the thickness of the material of the article of clothing 1. If the material is a woven fabric, the increase in the thickness of the material can be achieved by using threads of larger diameter to produce the fabric in the area of the strips; it is also possible to use a larger number of threads in the area of the strips 3, which again has the effect of increasing the thickness of the material.

The strips 3 have an approximately triangular cross section, so that a top or tip 31 is produced, which is contact with the skin 2. The sides 32, which extend toward the base 33 of the strip 3, proceed from the top or tip 31. In the present exemplary embodiment, the base 33 simultaneously forms the transition to the rest of the material of the article of clothing 1, designed here as a knee stocking.

The material of the article of the clothing 1 is made of the elastically resilient materials normally used for compression clothing. The material is elastic in all directions X, Y, and Z. The material exerts pressure in various directions, including the direction toward the skin 2. As a result of the regions of increased material thickness formed by the strips 3, the compressive force of the material exerted toward the skin 2 causes the tops or tips 31 to be pressed onto the skin 2, as indicated in FIG. 3 by the wavy configuration of the skin 2.

In FIGS. 12 and 13, the compression means are formed by fabric bands 8, which have an elasticity different from that of the base fabric of the article of clothing. Soft fabric bands thus alternate with hard fabric bands, which results in different compressive effects. The base fabric of the article of clothing used between adjacent fabric bands 8 has no compressive effect. The alternation between fabric bands with a compressive effect and bands without such an effect produces a regional type of compression.

The compression means 3, 8 can comprise interruptions 34, 81 (see FIGS. 5, 6, 8, 9, 11, and 13). This leads to a further improvement in the supply of “cooled” blood to the stressed muscles. That is, blood can circulate unhindered in the areas of the interruptions 34, 81; this is also possible in the areas adjacent to the compression means 3, 8.

The compression means 3, 8 can be oriented in almost any desired way. By way of example, FIGS. 4-13 show various possible orientations. In addition to the simple vertical orientation shown in FIGS. 4-6, a horizontal orientation (see FIGS. 7-9 and 12, 13) is also possible. The compression means 3, 8 can even have a spiral orientation, as shown in FIGS. 10 and 11. Common to all orientations is that the areas with the compressive effect alternate with regions without compression. This results in the inventive regional compression. As can be seen from the figures, the interruptions 34, 81, which further improve the inventive effects, can be provided independently of the orientation of the means 3, 8.

FIGS. 14 and 15 show schematic diagrams which compare the regional compressive effect according to the invention with the uniform, two-dimensional compressive effect according to the prior art. The course of the compression in the skin 2 is indicated by the lines 4. It can be seen that the skin 2 is compressed only in the area of the strips 3 or fabric bands 8. In these areas, the skin remains pale, whereas, in the area between the strips 3 or the fabric bands 8, the skin 2 turns red. The reddening of the skin is evidence of greater circulation in the area between the strips 3 or fabric bands 8. This is caused by the expansion of the capillaries 5, which occurs during physical exertion.

Because the strips 3 or fabric bands 8 rest only by way of individual spots or lines on the skin 2, the capillaries 5 located under the skin 2 undergo hardly any compression, as can be seen in FIG. 14. In contrast, in the case of the articles of clothing known from the prior art with continuous two-dimensional areas of compression, the walls of the capillaries 5 are squeezed together (compare FIG. 15), which has the result of impairing circulation. It can be derived from the diagram of the compression according to the prior art shown in FIG. 15 that the effects of compression, as illustrated by the lines 4, extend uniformly, regardless of the presence or absence of capillaries 5. As a result, the walls of the capillaries 5 are squeezed together, which gives the capillaries 5 a flattened, oval cross section. Under the compression clothing according to the prior art, therefore, the blood circulation required to cool the organism cannot be maintained, which has a negative effect on an athlete's performance.

Because the capillaries 5 between the strips 3 or fabric bands 8 are not compressed, it remains possible for blood to circulate under the skin in these areas, and as a result an exchange of heat takes place, which cools the organism. The heat arising on the skin 2 can also be carried away through the tunnel-like formations designated by the reference number 6 present in the areas between adjacent strips 3, the skin 2, and the material of the article of clothing 1. The gap between the article of clothing 1 and the skin 2 in the area between the strips 3 also prevents the article of clothing from becoming soaked with perspiration. Instead, contact between the article of clothing 1 and the skin 2 exists only in the area of the tops or tips 31 of the strips 3, which means that the article of clothing can become wet only in this area. As a result, the inventive article of clothing becomes even more comfortable to wear.

The wearing comfort can also be increased by providing the strips 3 with a coating 7. Various types of coatings 7 are possible. For example, functional coatings can be used, which provide antifungicidal or antibacterial effects, such as coatings with a high percentage of gold or silver. But other types of coatings can also be used, such a coating of polytetrafluoroethylene. This material, which is known under the trade name “Teflon”, is characterized by its good sliding properties, among others. When this is used as a coating for the strips 3, the friction between the article of clothing 1 and the skin 2 is significantly reduced, which also contributes to an increase in wearing comfort.

Although the invention has been described above on the basis of a knee stocking by way of example, the invention is not to be considered limited in any way to this object. Instead, the invention can be applied to any article of clothing which can be used as an article of compressive clothing. This includes not only the knee stockings described above as the exemplary embodiment but also pantyhose, armlets, shirts, or overalls. These articles of compression clothing, too, are covered by the inventive idea.

1. Compression clothing to be worn on the skin, made of elastically resilient material, characterized in that compression means are provided in certain areas, and the areas with a compressive effect are arranged in alternation with areas without compression, said compression means being formed by strips (3), which are provided on the side facing the skin (2).

2. (canceled)

3. Compression clothing according to claim 1, characterized in that the strips (3) are produced by an increase in the thickness of the material.

4. Compression clothing according to claim 1, characterized in that the strips (3) have an approximately triangular form.

5. Compression clothing according to claim 1, characterized in that the strips (3) are arranged at regular intervals.

6. Compression clothing according to claim 1, characterized in that the strips (3) are provided with a coating (7).

7. Compression clothing according to claim 1, characterized in that the compression means are formed by fabric bands (8), the elasticity of which is different from that of the base fabric of the compression clothing.

8. Compression clothing according to claim 7, characterized in that the strips (3) and fabric bands (8) comprise interruptions (34, 81).

9. Compression clothing according to claim 7, characterized in that the strips (3) and fabric bands (8) are oriented horizontally.

10. Compression clothing according to claim 7, characterized in that the strips (3) and fabric bands (8) are oriented vertically.

11. Compression clothing according to claim 7, characterized in that the strips (3) and fabric bands (8) have a spiral orientation.

12. Compression clothing according to claim 3, characterized in that the strips (3) are arranged at regular intervals.

13. Compression clothing according to claim 3, characterized in that the strips (3) are provided with a coating (7).

14. Compression clothing according to claim 3, characterized in that the strips (3) and fabric bands (8) each comprise interruptions (34, 81), respectively.

15. Compression clothing according to claim 3, characterized in that the strips (3) and fabric bands (8) are oriented horizontally.

16. Compression clothing according to claim 3, characterized in that the strips (3) and the fabric bands (8) are oriented vertically.

17. Compression clothing to be worn on the skin, made of elastically resilient material, characterized in that compression means are provided in certain areas, and the areas with a compressive effect are arranged in alternation with areas without compression, said compression means being formed by strips (3), which are provided on the side facing the skin (2), said strips (3) being produced by an increase in the thickness of the material, and said strips (3) being arranged at regular intervals.
